(19) United States
(12) Patent Application Publication
(10) Pub. No.: US 2025/0017511 A1
(43) Pub. Date: Jan. 16, 2025

(54) ST ELEVATED MYOCARDIAL INFARCTION (STEMI) DETECTION IN INSERTABLE CARDIAC MONITOR

(71) Applicant: Cardiac Pacemakers, Inc., St. Paul, MN (US)

(72) Inventors: Abhijit Rajan, Shoreview, MN (US); David L. Perschbacher, Blaine, MN (US); Krzysztof Z. Siejko, Maple Grove, MN (US)

(21) Appl. No.: 18/767,560

(22) Filed: Jul. 9, 2024

Related U.S. Application Data
(60) Provisional application No. 63/526,092, filed on Jul. 11, 2023.

(51) Int. Cl. A61B 5/358; A61B 5/00; A61B 5/287; A61B 5/29

(52) U.S. Cl. CPC A61B 5/358; A61B 5/0006; A61B 5/287; A61B 5/29; A61B 5/686; A61B 5/7225; A61B 5/7253; A61B 5/7264; A61B 5/746; A61B 2560/045; A61B 2560/0462; A61B 2562/0204

(57) ABSTRACT

Systems and methods are disclosed to signal processing device of a patient management system. The signal processing device comprising a communication circuit configured to receive a cardiac signal from an AMD, wherein the received cardiac signal is a frequency filtered cardiac signal produced from a broader frequency band cardiac signal sensed using the AMD; signal processing circuitry configured to restore the received cardiac signal to the broader frequency band cardiac signal; detect an elevated ST segment in the restored broader frequency band cardiac signal; and generate an alert of myocardial infarction in response to detecting the elevated ST segment.

### CLAIM OF PRIORITY

This application claims the benefit of U.S. Provisional Application No. 63/526,092 filed on Jul. 11, 2023, which is hereby incorporated by reference in its entirety.

### TECHNICAL FIELD

This document relates generally to medical devices and more particularly to systems, methods, and devices to detect myocardial infarction.

### BACKGROUND

Ambulatory medical devices (AMDs), including implantable, subcutaneous, wearable, or one or more other medical devices, etc., can monitor, detect, or treat, various conditions including, among other things, heart failure (HF), fibrillation, and myocardial infarction (MI). Myocardial infarction is a condition caused by a reduced or complete cessation of blood supply to the myocardial tissue of the heart. Ambulatory medical devices can include sensors to sense physiological information from a patient and one or more circuits to detect one or more physiologic events using the sensed physiological information or transmit sensed physiologic information or detected physiologic events to one or more remote devices. Patient monitoring can provide early detection of worsening patient condition, including MI.

### SUMMARY

Systems and methods are disclosed to detect ST-segment elevation myocardial infarction (STEMI) of a patient. Pairing the STEMI detection with patient indicated symptoms (e.g., chest pain) increases the specificity of device-based detection of STEMI.

Example 1 includes subject matter such as a signal processing device of a patient management system comprising a communication circuit and a signal processing circuit. The communication circuit is configured to receive a cardiac signal from an AMD, wherein the received cardiac signal is a frequency filtered cardiac signal produced from a broader frequency band cardiac signal sensed using the AMD. The signal processing circuit is configured to restore the received cardiac signal to the broader frequency band cardiac signal, detect an elevated ST segment in the restored broader frequency band cardiac signal, and generate an alert of myocardial infarction in response to detecting the elevated ST segment.

In Example 2, the subject matter of Example 1 optionally includes signal processing circuitry configured to reverse filter the frequency filtered cardiac signal to produce the restored broader frequency band cardiac signal.

In Example 3, the subject matter of Example 1 optionally includes signal processing circuitry configured to reverse filter the frequency filtered cardiac signal to produce a reverse filtered signal, and phase correct the reverse filtered signal to produce the restored broader frequency band cardiac signal.

In Example 4, the subject matter of one or any combination of Examples 1-3 optionally includes a memory to store one or more baseline ST segment elevation measurements, and signal processing circuitry configured to compare elevation of the ST segment of the restored cardiac signal to the one or more baseline ST segment elevation measurements and detect that the ST segment of the restored cardiac signal is elevated based on the comparison.

In Example 5, the subject matter of one or any combination of Examples 1-4 optionally includes signal processing circuitry is included in a server of the patient management system.

In Example 6, the subject matter of one or any combination of Examples 1-5 optionally includes a communication circuit configured to receive a sensed heart sound signal from the AMD, and signal processing circuitry configured to detect an indication of myocardial infarction in the sensed heart sound signal and produce the alert when detecting the elevated ST segment in the restored broader frequency band cardiac signal and detecting the indication of myocardial infarction in the sensed heart sound signal.

In Example 7, the subject matter of one or any combination of Examples 1-6 optionally includes signal processing circuitry configured to produce a broader frequency band cardiac signal from a high pass filtered cardiac signal received from the AMD.

In Example 8, the subject matter of one or any combination of Examples 1-7 optionally includes a communication circuit configured to communicate information with the AMD during a communication session initiated by the AMD and receive the frequency filtered cardiac signal from the AMD during the communication session.

Example 9 includes subject matter (such as a method of operating a patient management system) or can optionally be combined with one or any combination of Examples 1-8 to include such subject matter, comprising receiving, by signal processing circuitry of the patient management system, a cardiac signal of a patient, wherein the received cardiac signal is a frequency filtered cardiac signal produced from a broader frequency band cardiac signal sensed using and AMD; restoring, using the signal processing circuitry, the received cardiac signal to the broader frequency band cardiac signal; and producing an alert of myocardial infarction when detecting an elevated ST segment in the restored broader frequency band cardiac signal.

In Example 10, the subject matter of Example 9 optionally includes the signal processing circuitry reverse filtering the frequency filtered cardiac signal to produce the restored broader frequency band cardiac signal.

In Example 11, the subject matter of Example 9 optionally includes the signal processing circuitry reverse filtering the frequency filtered cardiac signal to produce a reverse filtered signal and phase correcting the reverse filtered signal to produce the restored broader frequency band cardiac signal.

In Example 12, the subject matter of one or any combination of Examples 9-11 optionally includes comparing the ST segment of the restored cardiac signal to one or more baseline ST segments for the patient and detecting that the ST segment of the restored cardiac signal is elevated from the one or more baseline ST segments.

In Example 13, the subject matter of one or any combination of Examples 9-12 optionally includes receiving, by the signal processing circuitry, a heart sound signal sensed using the AMD; detecting, by the signal processing circuitry, myocardial infarction using the sensed heart sound signal; and producing the alert when detecting the elevated ST segment in the restored broader frequency band cardiac signal and detecting the myocardial infarction using the sensed heart sound signal

In Example 14, the subject matter of one or any combination of Examples 9-13 optionally includes initiating sensing of the broader frequency band cardiac signal by the AMD in response to a patient trigger received by the AMD.

In Example 15, the subject matter of one or any combination of Examples 9-14 optionally includes receiving a high pass filtered cardiac signal having lower frequency signal components removed from the sensed broader frequency band cardiac signal, and restoring the lower frequency signal components in the cardiac signal.

In Example 16, the subject matter of one or any combination of Examples 9-15 optionally includes the signal processing circuitry of a remote device remote from the AMD receiving the frequency filtered cardiac signal from the AMD via a communication link, and restoring the received cardiac signal to the broader frequency band cardiac signal.

In Example 17, the subject matter of Example 16 optionally includes the signal processing circuitry of a server receiving the frequency filtered cardiac signal from the AMD via the communication link.

In Example 18, the subject matter of one or both of Examples 16 and 17 optionally includes sending, by the AMD, the frequency filtered cardiac signal from the AMD to the remote device during a communication session initiated by the AMD in response to a patient trigger.

Example 19 includes subject matter (or can optionally be combined with one or any combination of Examples 1-18 to include such subject matter) such as a computer readable storage medium including instructions that when performed by one or more processors of a medical device cause the medical device to perform operations including receiving, by signal processing circuitry of the patient management system, a cardiac signal of a patient, wherein the received cardiac signal is a frequency filtered cardiac signal produced from a broader frequency band cardiac signal sensed using an ambulatory medical device (AMD); restoring, using the signal processing circuitry, the received cardiac signal to the broader frequency band cardiac signal; and producing an alert of myocardial infarction when detecting an elevated ST segment in the restored broader frequency band cardiac signal.

In Example 20, the subject matter of Example 19 optionally includes instructions that when performed by the one or more processors of the medical device cause the medical device to perform operations including reverse filtering the frequency filtered cardiac signal to produce a reverse filtered signal, and phase correcting the reverse filtered signal to produce the restored broader frequency band cardiac signal.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example patient management system.

FIG. 2 illustrates an example of an ambulatory medical device (AMD) that is an implantable medical device.

FIG. 3 is a block diagram of portions of electronic circuits of an AMD.

FIG. 4 is a block diagram of portions of an external device included in a patient management system.

FIG. 5 is an illustration of a PQRST complex of an electrocardiogram.

FIGS. 6A-6B are illustrations of strip chart recordings of an electrocardiogram.

FIG. 7 is a flow diagram of an example of operating a patient management system.

FIG. 8 is an illustration of reverse filtering of an electrical cardiac signal.

FIG. 9 illustrates a block diagram of an example machine upon which any one or more of the techniques discussed herein may perform.

### DETAILED DESCRIPTION

Ambulatory medical devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

The present inventors have recognized, among other things, systems, and methods to provide device-based detection of ST-segment elevation myocardial infarction (STEMI) of a patient. The device-based monitoring can improve the time to intervention to address the STEMI.

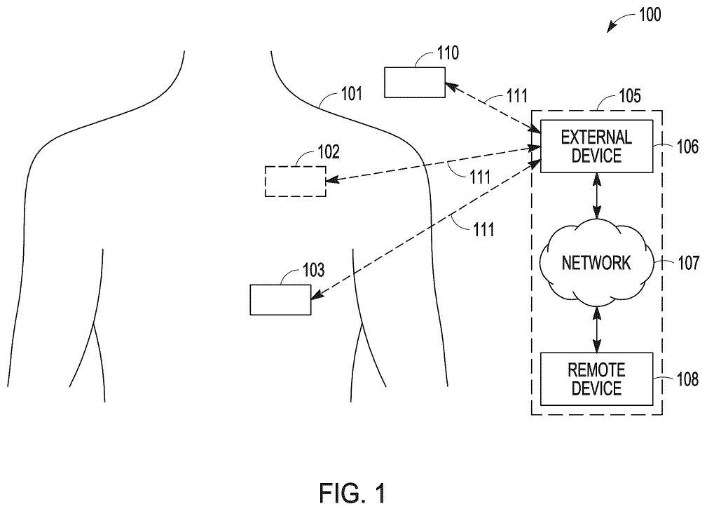

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more medical devices, an external system 105, and a communication link 111 providing for communication between the one or more ambulatory medical devices and the external system 105. The one or more medical devices can include an ambulatory medical device (AMD), such as an implantable medical device (IMD) 102, insertable cardiac monitor (ICM), a wearable medical device 103, or one or more other implantable, leadless, subcutaneous, external, wearable, or medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 of FIG. 1 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes, such as described herein. The implantable medical device 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the implantable medical device 102 or one or more of the other ambulatory medical devices, etc. In some examples, therapy can include CRT for rectifying dyssynchrony and improving cardiac function in heart failure patients. In other examples, the implantable medical device 102 can include a drug delivery system, such as a drug infusion pump to deliver drugs to the patient for managing arrhythmias or complications from arrhythmias, hypertension, hypotension, or one or more other physiologic conditions. In other examples, the implantable medical device 102 can include one or more electrodes configured to stimulate the nervous system of the patient or to provide stimulation to the muscles of the patient airway, etc.

The wearable medical device 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the implantable medical device 102 or one or more other ambulatory medical devices connected to the external system 105 via a communication link 111. In other examples, the IMD 102 can be connected to the wearable medical device 103, or the wearable medical device 103 can be connected to the external system 105, via the communication link 111. This can include, for example, programming the IMD 102 to perform one or more of acquiring physiologic data, performing at least one self-diagnostic test (such as for a device operational status), analyzing the physiologic data, or optionally delivering or adjusting a therapy for the patient 101. Additionally, the external system 105 can send information to, or receive information from, the IMD 102 or the wearable medical device 103 via the communication link 111. Examples of the information can include real-time or stored physiologic data from the patient 101, diagnostic data, such as detection of patient hydration status, hospitalizations, responses to therapies delivered to the patient 101, or device operational status of the implantable medical device 102 or the wearable medical device 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or both of the external device 106 and the remote device 108 can output the detected medical events to a system user, such as the patient or a clinician, or to a process including, for example, an instance of a computer program executable in a microprocessor or other processor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

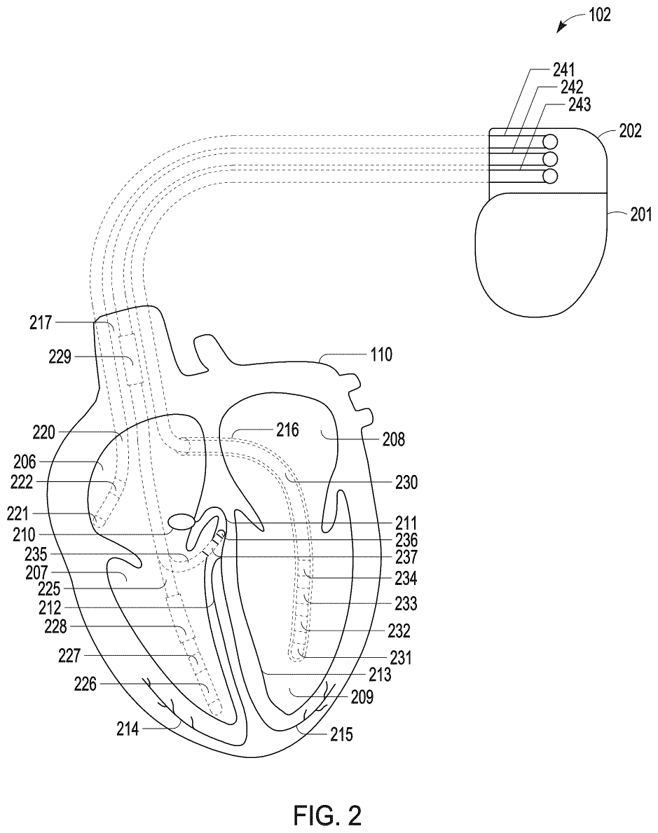

FIG. 2 illustrates an example of an AMD that is an IMD 102. The IMD 102 is electrically coupled to a heart 110, such as through one or more leads coupled to the IMD 102 through one or more lead ports, such as first, second, or third lead ports 241, 242, 243 in a header 202 of the IMD 102. In an example, the IMD 102 can include an antenna, such as in the header 202, configured to enable communication with an external system and one or more electronic circuits in a hermetically sealed housing (CAN) 201. The IMD 102 illustrates an example medical device (or a medical device system) as described herein.

The IMD 102 may be an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the IMD 102 can include one or more electrodes or other sensors (e.g., a pressure sensor, an accelerometer, a gyroscope, a microphone, etc.) powered by a power source in the IMD 102. The one or more electrodes or other sensors of the leads, the IMD 102, or a combination thereof, can be configured detect physiologic information from, or provide one or more therapies or stimulation to, the patient.

The IMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

In an example, the IMD 102 can sense impedance such as between electrodes located on one or more of the leads or the CAN 201. The IMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 102 can be configured to inject current between an electrode on one or more of the first, second, third, or fourth leads 220, 225, 230, 235 and the CAN 201, and to sense the resultant voltage between the same or different electrodes and the CAN 201.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

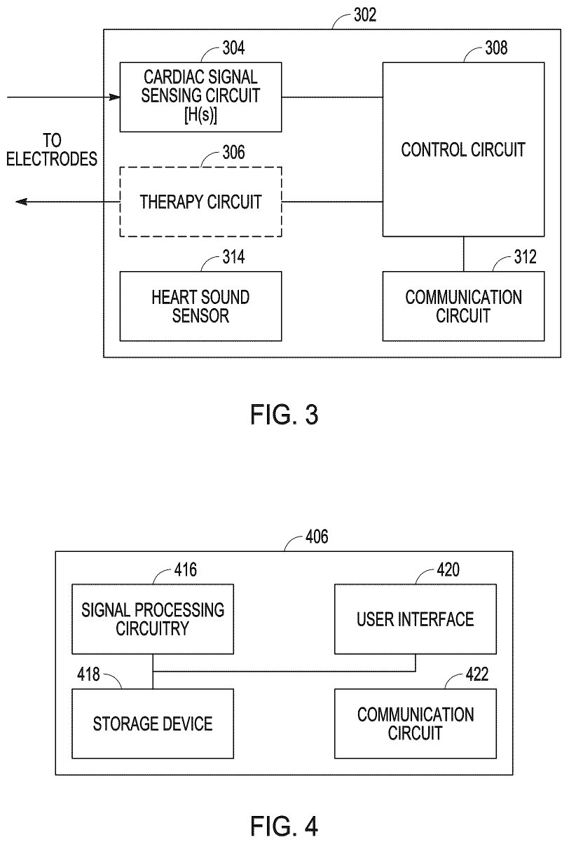

FIG. 3 is a block diagram of portions of electronic circuits of an AMD 302 that is implantable. The AMD 302 can be coupled to multiple implantable electrodes, such as the electrode arrangement described in the example of FIG. 2. The AMD 302 includes a cardiac signal sensing circuit 304, a therapy circuit 306, a communication circuit 312, and a control circuit 308. The communication circuit 312 can be used to communicate information wirelessly with a separate device. The therapy circuit 306 provides electrical pacing stimulation energy to the heart of the patient when operatively connected to pacing electrodes of the system. The pacing electrodes can include any of the pacing electrodes in FIG. 2, such as electrodes configured placement in or near the RA, RV, LV, His Bundle, or left bundle branches, and an electrode of the CAN 201.

The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by signal processing circuitry 310 of the control circuit 308. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc. Far-field cardiac signals can be sensed using the electrode of the CAN. In some examples, the AMD 302 is a diagnostic-only device (e.g., an ICM) and does not include a therapy circuit 306.

The control circuit 308 may include a digital signal processor, application specific integrated circuit (ASIC), field programmable gate array (FPGA), microprocessor, or other type of processor, interpreting or executing instructions in software or firmware. In some examples, the control circuit 308 may include a state machine or sequencer that is implemented in hardware circuits. The control circuit 308 may include any combination of hardware, firmware, or software. The control circuit 308 includes electronic circuitry (e.g., signal processing circuitry 310) to perform the functions described herein. A circuit may include software, hardware, firmware or any combination thereof. For example, the circuit may include instructions in software executing on the control circuit 308. Multiple functions may be performed by one or more circuits of the control circuit 308.

FIG. 4 is a block diagram of a device 406 included in an external system (e.g., external system 105 in FIG. 1). The device 406 may be either an external device of the external system (e.g., external device 106 in FIG. 1) or a remote device of the external system (e.g., remote device 108 in FIG. 1). The device 406 also includes a storage device 418 and signal processing circuitry 416. In some examples the device includes a user interface 420. Signal processing circuitry 416 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or portions thereof, and a programmable logic circuit or a portion thereof. The storage device 418 may be a memory integral to the and signal processing circuitry 416, or a separate memory device.

In some examples, the device 406 includes a communication circuit 422 to communicate information with another device. Communication circuit 422 may communicate information wirelessly with the AMD 302 of FIG. 3 using near-field inductive wireless signals or far-field radio-frequency signals. The device 406 can be used to program pacing therapy parameters and other information in the AMD 302. In some examples, the device 406 is remote from the AMD 302 and the communication circuit 422 communicates information over a network (e.g., a cellular phone network or the Internet) to another device that communicates information wirelessly with the AMD 302.

Myocardial infarction (MI) is caused by blockage in the coronary arteries. This blockage leads to decreased of complete cessation of blood flow to a portion of the myocardia. Symptoms of MI include chest pain, fatigue, and shortness of breath. There are two types of MI: ST-segment elevation myocardial infarction (STEMI) and Non-ST-segment elevation myocardial infarction (NSTEMI).

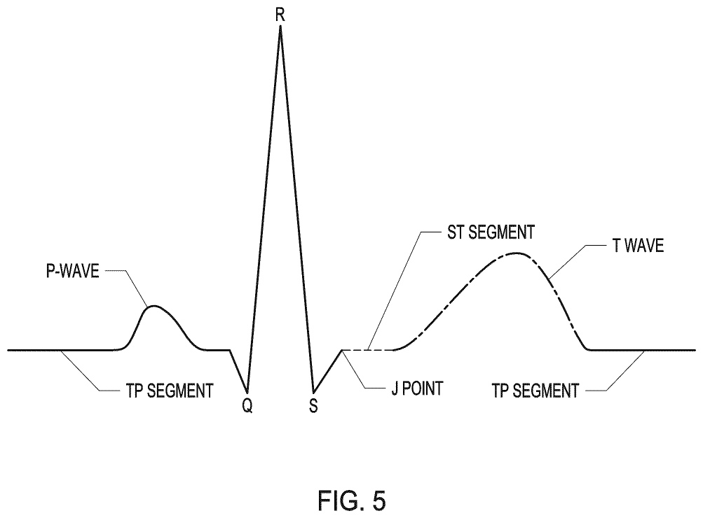

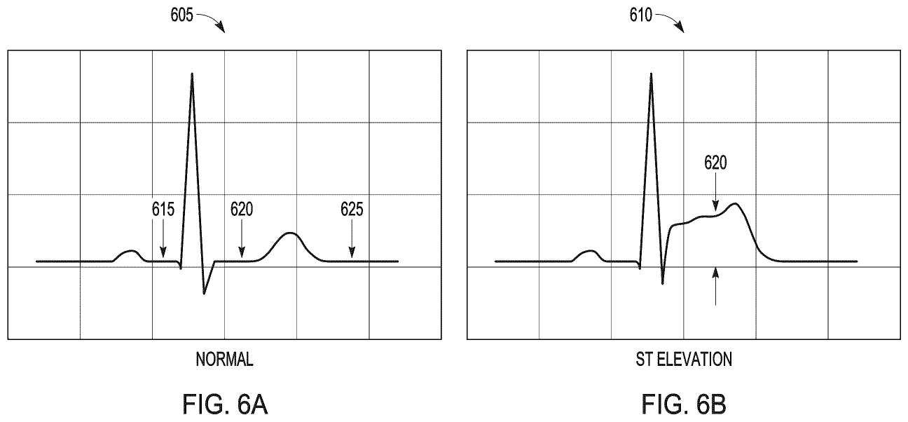

FIG. 5 is an illustration of a PQRST complex of an electrocardiogram (ECG), including the S-wave and T-wave, and the ST segment. In MI, the changes in the ST segment are produced by the flow of currents referred to as “injury currents” generated between the ischemic and non-ischemic myocardial tissue. FIGS. 6A and 6B are illustrations of strip chart recordings of an ECG 605 with a normal ST segment, and an ECG 610 with an elevated ST segment. For individuals without prior cardiac disease as in ECG 605 of FIG. 6A, the level of the ST segment 620 is substantially the same as the level of the PR segment 615 or the level of the following the TP segment 625 (i.e., the isoelectric or plateau phase). In ECG 610 of FIG. 6B, the ST segment 620 is elevated above isoelectric or plateau level. Coupled with the symptom of chest pain of the patient, system-based detection of an elevated ST segment can improve STEMI detection.

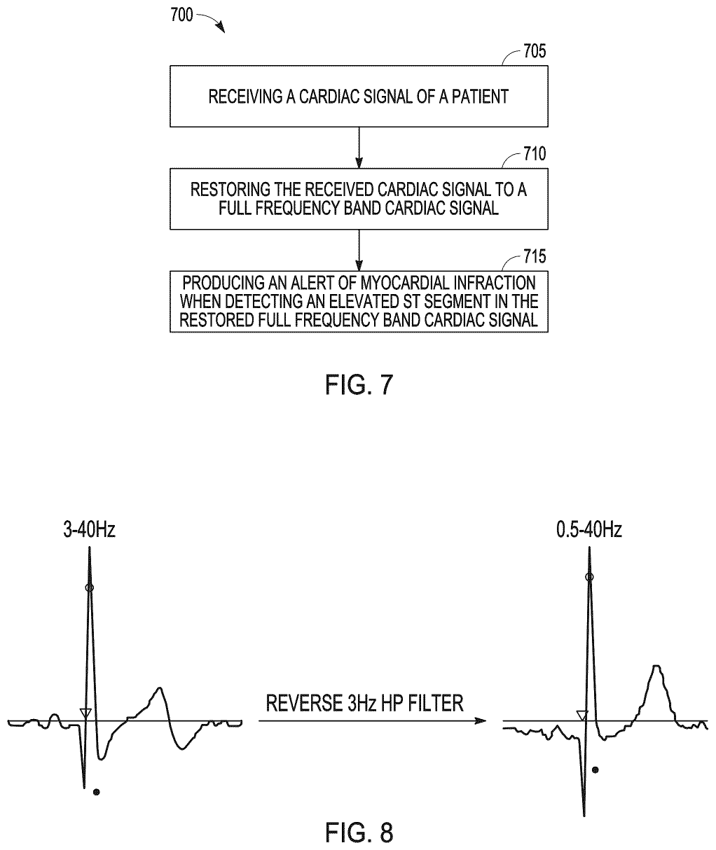

FIG. 7 is a flow diagram of an example of a method 700 of operating a patient management system (e.g., the patient management system 100 of FIG. 1). At block 705, the signal processing circuitry of the patient management system (e.g., signal processing circuitry 416 in FIG. 4) receives a sensed cardiac signal of the patient. The cardiac signal may be sensed using a cardiac signal sensing circuit of an AMD of the patient management system (e.g., cardiac signal sensing circuit 304 in FIG. 3). The signal processing circuitry may be included in an external device and receives the sensed cardiac signal from the AMD through a wireless communication link, or the signal processing circuitry may be included in a remote device (e.g., a remote server) and receives the sensed cardiac signal from a device different from the AMD (e.g., from an external device).

The AMD may begin sampling and recording the cardiac signal in response to a trigger from the patient. For instance, the patient may trigger the recording of one or more cardiac signals in response to chest pain. A patient may have a personal device (e.g., a handheld communicator or a smartphone) that can communicate with the AMD. The personal device may run an application (or App) that allows the patient to send the trigger to the AMD. In variations, the AMD is a wearable medical device, and the patient can push a button to trigger signal recording or interact in another way with a user interface of the wearable medical device to trigger the signal recording.

The AMD may initiate a communication session with the external device to send the recorded signal or signals to the external device. In certain examples, the patient trigger is received by the external device, and the external device initiates a communication session with the AMD to send a command to the AMD to begin recording the cardiac signal and sending the recorded cardiac signal to the external device.

The cardiac signal sensed by the AMD is a frequency filtered cardiac signal produced from a raw signal that is unfiltered and unamplified. For example, the cardiac signal sensed by the AMD may be high pass filtered or bandpass filtered. In certain examples, the cardiac signal sensed by the AMD is band limited to a 3-40 Hertz (3-40 Hz) signal. The AMD records the cardiac signal for a predetermined duration of time (e.g., multiple minutes) and send the recording to the signal processing circuitry of the patient management system.

At block 710, the signal processing circuitry restores the received cardiac signal to the broader frequency band of the raw cardiac signal. In some examples, the signal processing circuitry performs reverse filtering on the filtered cardiac signal to produce the restored broader frequency band cardiac signal. For instance, the amplifier circuits and filter circuits of the cardiac signal sensing circuit 304 in FIG. 3 may apply a known transfer function (e.g., H(s)) to the input to the cardiac signal sensing circuit 304 to produce the frequency filtered cardiac signal at the output. The signal processing circuitry of the patient management system may process the frequency filtered cardiac signal with the inverse of the transfer function (H⁻¹(s)) to produce the restored broader frequency band cardiac signal. In certain examples, a 3-40 Hz cardiac signal sensed by the AMD is restored to a 0.5-40 Hz signal by the signal processing circuitry of the patient management system. Thus, the lower frequencies of the raw signal are restored.

When the cardiac signal from the AMD is restored, the signal processing circuitry then analyses the restored cardiac signal for an elevated ST segment. A cardiac signal closer to the raw signal is produced and analyzed to ensure that any elevated ST segment is physiological and not a product of the filtering by the AMD. FIG. 8 is an illustration of a 3-40 Hz reverse filtered to produce a 0.5-40 Hz signal. It can be seen in FIG. 8 that reverse filtering to restore lower frequencies affects the ST segment.

In some examples, the reverse filtered signal is phase corrected by the signal processing circuitry to produce the restored broader frequency band cardiac signal. The cardiac signal sensing circuit may cause phase distortion when processing the raw signal input to the cardiac signal sensing circuit. The signal processing circuitry may phase correct the cardiac signal. For instance, the signal processing circuitry may invert or flip the reverse filtered signal and process the flipped signal with the transfer function of the cardiac signal sensing circuit of the AMD.

After restoring the cardiac signal, the signal processing circuitry checks the restored cardiac signal for an ST segment elevation. According to some examples, signal processing circuitry is operatively connected to a storage device (e.g., the storage device 418 in FIG. 4) that stores one or more baseline cardiac signals previously recorded for the patient or measurements of elevation of the ST segments of the baseline signals. The signal processing circuitry compares the ST segment of the restored broader frequency band cardiac signal to one or more ST segments of the baseline signals recorded for the patient. The signal processing circuitry may detect that the ST segment is elevated when the amplitude for the ST segment is higher than the baseline ST segment or segments by more than a predetermined (e.g., programmed) threshold amplitude.

Returning to FIG. 7 at block 715, the device with the signal processing circuitry produces an alert of myocardial infarction when detecting an elevated ST segment in the restored broader frequency band cardiac signal. One or more alerts can be provided, such as to the patient, to a clinician, or to one or more other caregivers (e.g., using a patient smart watch, a cellular or smart phone, a computer, etc.) in response to the detection. The alerts may instruct the caregivers to take some action such as providing anti-coagulants to the patient or to start reperfusion therapy.

As explained previously herein, detection of an elevated ST segment coupled with the symptom of chest pain of the patient can improve specificity of STEMI detection. The specificity in STEMI detection may be improved by adding a system-based analysis of heart sounds of the patient. Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4).

Valve closures and blood movement and pressure changes in the heart can cause accelerations, vibrations, or movement of the cardiac walls that can be detected using a heart sound sensor such as an accelerometer or a microphone, producing a heart sound signal. In an example, heart sound signal portions, or values of respective heart sound signals for a cardiac interval, may be detected by comparison with a sensed cardiac signal. For instance, the value and timing of an S1 signal can be detected using an amplitude or energy of the heart sound signal occurring at or about the R wave of the cardiac interval. The S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles. For example, a heart sound parameter can include a composite S1 parameter representative of a plurality of S1 parameters, for example, over a certain time period (e.g., a number of cardiac cycles, a representative time period, etc.). In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform.

The AMD can include a heart sound sensor (e.g., heart sound sensor 314 in FIG. 3) to produce a heart sound signal. The AMD may record the heart sound signal in response to the patient trigger. The signal processing circuitry of the patient management system receives a recorded frequency filtered cardiac signal and a recorded heart sound signal from the AMD, and detects STEMI using the restored broader frequency band cardiac signal and the heart sound signal.

For example, the signal processing circuitry may detect STEMI when detecting an elevated ST segment in restored cardiac signal and detecting that an S4 heart sound begins appearing in the heart sound signal. In another example, the signal processing circuitry may detect STEMI when detecting an elevated ST segment in restored cardiac signal and detecting that intensities of the S1 and S2 heart sounds decrease and one or both of an S3 heart sound and an S4 heart sound start appearing in the heart sound signal. The alert can be produced when detecting both the elevated ST segment in the restored broader frequency band cardiac signal and detecting an indication of MI in the sensed heart sound signal.

The AMD may transition from a lower power mode to a higher power mode when collecting the ST segment information and the heart sound information. The higher power mode can include one or more of: enabling one or both of a cardiac signal sensing circuit and a heart sound sensing circuit, increasing a sensing frequency or a sensing or storage resolution, increasing an amount of data to be collected, communicated (e.g., to a second medical device, etc.), or stored, triggering storage of currently available information or increasing the storage capacity or time period of a loop recorder, or otherwise altering device behavior to capture additional or higher-resolution physiologic information or perform more processing, etc. After the sensing, recording, and transmitting of information, the AMD returns to the lower power mode.

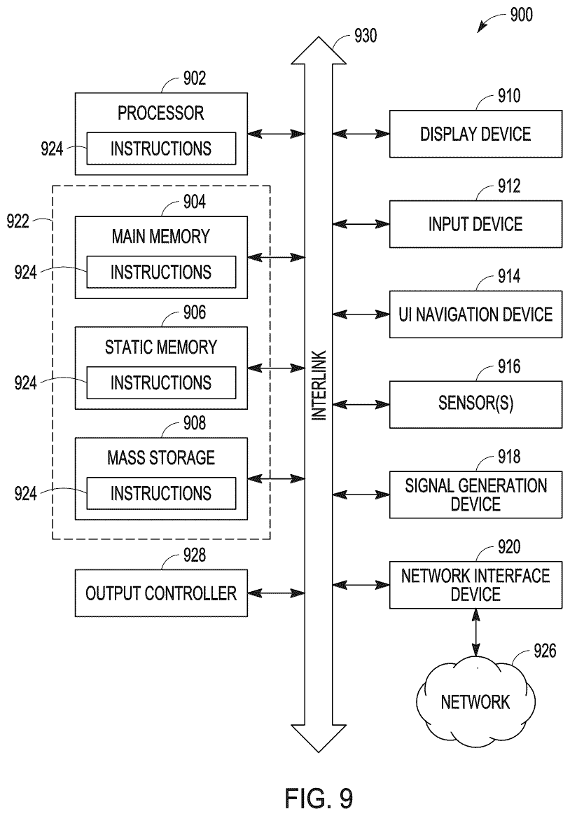

FIG. 9 illustrates a block diagram of an example machine 900 upon which any one or more of the techniques (e.g., methodologies) discussed herein may perform. Portions of this description may apply to the computing framework of one or more of the medical devices described herein, such as the ambulatory medical device, the external programmer, the remoter server, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 900. Circuitry (e.g., signal processing circuitry, etc.) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 900 (e.g., computer system) may include a hardware processor 902 (e.g., a central processing unit (CPU), a graphics processing unit (GPU), a hardware processor core, or any combination thereof), a main memory 904, a static memory 906 (e.g., memory or storage for firmware, microcode, a basic-input-output (BIOS), unified extensible firmware interface (UEFI), etc.), and mass storage 908 (e.g., hard drive, tape drive, flash storage, or other block devices) some or all of which may communicate with each other via an interlink 930 (e.g., bus). The machine 900 may further include a display unit 910, an input device 912 (e.g., a keyboard), and a user interface (UI) navigation device 914 (e.g., a mouse). In an example, the display unit 910, input device 912, and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or one or more other sensors. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the hardware processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable medium 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 900, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A signal processing device of a patient management system, the device comprising:
a communication circuit configured to receive a cardiac signal from an AMD, wherein the received cardiac signal is a frequency filtered cardiac signal produced from a broader frequency band cardiac signal sensed using the AMD; and
signal processing circuitry configured to:
restore the received cardiac signal to the broader frequency band cardiac signal;
detect an elevated ST segment in the restored broader frequency band cardiac signal; and
generate an alert of myocardial infarction in response to detecting the elevated ST segment.

2. The device of claim 1, wherein the signal processing circuitry is configured to reverse filter the frequency filtered cardiac signal to produce the restored broader frequency band cardiac signal.

3. The device of claim 1, wherein the signal processing circuitry is configured to:
reverse filter the frequency filtered cardiac signal to produce a reverse filtered signal; and
phase correct the reverse filtered signal to produce the restored broader frequency band cardiac signal.

4. The device of claim 1, including:
a memory to store one or more baseline ST segment elevation measurements; and
wherein the signal processing circuitry is configured to:
compare elevation of the ST segment of the restored cardiac signal to the one or more baseline ST segment elevation measurements; and
detect that the ST segment of the restored cardiac signal is elevated based on the comparison.

5. The device of claim 1, wherein the signal processing circuitry is included in a server of the patient management system.

6. The device of claim 1,
wherein the communication circuit is configured to receive a sensed heart sound signal from the AMD; and
wherein the signal processing circuitry is configured to:
detect an indication of myocardial infarction in the sensed heart sound signal; and
produce the alert when detecting the elevated ST segment in the restored broader frequency band cardiac signal and detecting the indication of myocardial infarction in the sensed heart sound signal.

7. The device of claim 1, wherein the signal processing circuitry is configured to produce a broader frequency band cardiac signal from a high pass filtered cardiac signal received from the AMD.

8. The device of claim 1, wherein the communication circuit is configured to:
communicate information with the AMD during a communication session initiated by the AMD; and
receive the frequency filtered cardiac signal from the AMD during the communication session.

9. A method of operating a patient management system, the method comprising:
receiving, by signal processing circuitry of the patient management system, a cardiac signal of a patient, wherein the received cardiac signal is a frequency filtered cardiac signal produced from a broader frequency band cardiac signal sensed using an ambulatory medical device (AMD);
restoring, using the signal processing circuitry, the received cardiac signal to the broader frequency band cardiac signal; and
producing an alert of myocardial infarction when detecting an elevated ST segment in the restored broader frequency band cardiac signal.

10. The method of claim 9, wherein the restoring the received cardiac signal to the broader frequency band cardiac signal includes the signal processing circuitry reverse filtering the frequency filtered cardiac signal to produce the restored broader frequency band cardiac signal.

11. The method of claim 9, wherein the restoring the received cardiac signal to the broader frequency band cardiac signal includes the signal processing circuitry reverse filtering the frequency filtered cardiac signal to produce a reverse filtered signal, and phase correcting the reverse filtered signal to produce the restored broader frequency band cardiac signal.

12. The method of claim 9, wherein the detecting the elevated ST segment in the restored broader frequency band cardiac signal includes:
comparing the ST segment of the restored cardiac signal to one or more baseline ST segments for the patient; and
detecting that the ST segment of the restored cardiac signal is elevated from the one or more baseline ST segments.

13. The method of claim 9, including:
receiving, by the signal processing circuitry, a heart sound signal sensed using the AMD;
detecting, by the signal processing circuitry, myocardial infarction using the sensed heart sound signal; and
wherein the producing the alert of myocardial infarction includes producing the alert when detecting the elevated ST segment in the restored broader frequency band cardiac signal and detecting the myocardial infarction using the sensed heart sound signal.

14. The method of claim 9, including:
initiating sensing of the broader frequency band cardiac signal by the AMD in response to a patient trigger received by the AMD.

15. The method of claim 9,
wherein the receiving the cardiac signal includes receiving a high pass filtered cardiac signal having lower frequency signal components removed from the sensed broader frequency band cardiac signal; and
wherein the restoring the received cardiac signal to the broader frequency band cardiac signal includes restoring the lower frequency signal components in the cardiac signal.

16. The method of claim 9,
wherein the receiving cardiac signal by the signal processing circuitry includes the signal processing circuitry of a remote device remote from the AMD receiving the frequency filtered cardiac signal from the AMD via a communication link; and
wherein the restoring the received cardiac signal includes the signal processing circuitry of the remote device restoring the received cardiac signal to the broader frequency band cardiac signal.

17. The method of claim 16, wherein the receiving cardiac signal by the signal processing circuitry includes the signal processing circuitry of a server receiving the frequency filtered cardiac signal from the AMD via the communication link.

18. The method of claim 16, including sending, by the AMD, the frequency filtered cardiac signal from the AMD to the remote device during a communication session initiated by the AMD in response to a patient trigger.

19. A non-transitory computer readable storage medium including instructions that when performed by one or more processors of a medical device cause the medical device to perform operations including:
receiving, by signal processing circuitry of the patient management system, a cardiac signal of a patient, wherein the received cardiac signal is a frequency filtered cardiac signal produced from a broader frequency band cardiac signal sensed using an ambulatory medical device (AMD);
restoring, using the signal processing circuitry, the received cardiac signal to the broader frequency band cardiac signal; and
producing an alert of myocardial infarction when detecting an elevated ST segment in the restored broader frequency band cardiac signal.

20. The non-transitory computer readable storage medium of claim 19, including instructions that when performed by the one or more processors of the medical device cause the medical device to perform operations including:
reverse filtering the frequency filtered cardiac signal to produce a reverse filtered signal; and
phase correcting the reverse filtered signal to produce the restored broader frequency band cardiac signal.
